(19) United States
(12) Patent Application Publication
(10) Pub. No.: US 2009/0069694 A1
(43) Pub. Date: Mar. 12, 2009

(54) Coronary sinus access catheter with forward-imaging means

(76) Inventors: David Amundson, Boulder, CO (US); H. John Hanlin, Louisville, CO (US); Larry Blankenship, Boulder, CO (US)

Correspondence Address: PETER F WEINBERG;GIBSON DUNN AND CRUTCHER LLP, SUITE 4100, 1801 CALIFORNIA STREET, DENVER, CO, 80202, US

(21) Appl. No.: 12/286,850

(22) Filed: Oct. 2, 2008

Related U.S. Application Data
(63) Continuation of application No. 10/495,036, filed on May 10, 2004, now abandoned.

(30) Foreign Application Priority Data
Nov. 12, 2002 (US) PCT/US02/36191

(51) Int. Cl. A61B 8/14; A61B 1/04

(52) U.S. Cl. 600/467; 600/109

(57) ABSTRACT

A coronary access catheter system simplifies the insertion of objects into distal branches of the coronary sinus. The system incorporates a real-time forward-imaging means to view the os and the branches of the coronary sinus. Preferably, the catheter uses near-infrared light as the forward-imaging means, but it could also include ultrasound or electromagnetic transducer. As the image is viewed, the catheter tip can be steered into the coronary sinus os and deflected in a tight radius bend on the distal end to navigate the short radius, right angle turns found in the coronary sinus branches. At that point, a flexible sheath can be placed over the guide catheter or objects such as guidewires can be inserted into channels of the guide catheter. The system consists of a catheter and image acquisition unit, which displays the forward image.

This application claims the benefit of U.S. provisional patent application No. 60/332,654 filed on Nov. 9, 2002 and patent application Ser. No. 10/495,036 filed May 10, 2004.

### BACKGROUND OF THE INVENTION

1. Field of the Invention

This invention relates to cardiac catheters/introducers used to access the coronary sinus and navigate the sinus vasculature using a deflecting distal end with feedback provided by a forward-imaging means.

2. Related Art

The following references provide useful information in the filed of the present invention, and are incorporated by reference herein:

| | | |
|---|---|---|
| Lurie | 6/1995 | 5,423,772 |
| Adams | 7/19955 | 433,729 |
| Jaraczewski | 8/1995 | 5,445,148 |
| Toner | 2/1996 | 5,488,960 |
| Avitall | 7/1997 | 5,642,736 |
| Swoyer | 11/1997 | 5,683,445 |
| Randolph | 7/1998 | 5,775,327 |
| Wang | 3/2000 | 6,041,248 |
| Tockman | 10/2000 | 6,129,750 |
| Amundson | 1/2001 | 6,178,346 |
| Lin | 3/2001 | 6,200,269 |
| Williams | 6/2002 | 6,408,214B1 |
| Zeylikovich | 8/2002 | 6,437,867 |
| Ockuly | 10/2002 | 6,458,107B1 |

Cardiac catheterizations are procedures in which a cardiologist inserts a catheter in the venous or arterial systems and navigates to the site of interest such as an artery, vein or chambers of the heart. In recent years, there has been increased interest in navigating the coronary sinus vasculature, particularly for the placement of permanent cardiac pacing and defibrillation leads intended to pace or defibrillate the left ventricle. A new modality called biventricular pacing, has been developed which paces both the left and right ventricles as well as the right atrium to insure synchrony of the right and left ventricular contraction. One-third of patients with chronic heart failure have electrocardiographic evidence of a major intraventricular conduction delay, which may worsen left ventricular systolic dysfunction through asynchronous ventricular contraction. Uncontrolled studies suggest that biventricular pacing improves hemodynamics and well-being by reducing ventricular asynchrony. Biventricular pacing is accomplished by using a lead inserted in the coronary sinus to pace the left ventricle.

The coronary sinus vasculature wraps around the heart, with many branches lying laterally on the left ventricle in close proximity to ventricular muscle fibers. It is thus possible to pace or defibrillate these fibers in the left ventricle through an electrically conductive lead inserted from the right side of the heart. Permanent endocardial leads or other devices are only placed in the right heart, since implanted objects produce an inflammatory response from the body, frequently with some thrombi formation. On the right side of the heart, a thrombus that has broken loose will travel to the lungs, with no deleterious effects. A thrombi formed in the left heart can travel to the brain, possibly producing a stroke. Consequently, pacemaker and defibrillator leads are implanted on the right side exclusively.

The coronary sinus vasculature is the gateway to the left ventricle, while still residing in the right heart circulatory system. Besides as a site for cardiac leads, the coronary sinus vasculature has been considered as a site for other therapeutic devices since branches span most of the left ventricle. For example, life-threatening, rapid heart rates can be treated by infusing ethanol alcohol in a coronary sinus branch in close proximity to tissue responsible for the condition. Alcohol contact results in cellular death and the discontinuation of the rapid heart rate. Heart cells can be rejuvenated as well by the infusion of drugs and cells, which augment the healing of infarcted heart cells. In this treatment, drugs or cells would be infused not a coronary sinus branch in close proximity to the infracted area. Also on the horizon, are genetic therapies in which DNA is seeded into specific sites in the ventricles to improve mechanical contractility. DNA in the form of naked DNA or DNA carried by a virus can be introduced via the coronary sinus vasculature to a coronary sinus distal branch close to the desired treatment point in the ventricles.

The coronary sinus vasculature is a venous system entered from a small hole (coronary sinus ostium or os) located on the tricuspid valve plane in the right atrium. The navigation inside the coronary sinus towards the left ventricle is complex, involving several sharp turns. Moreover, unlike the coronary artery system, blood is flowing towards the catheter. In the coronary artery system, navigation within the vasculature is based on infusing a radio opaque dye which flows downstream, elucidating the branch points ahead of the catheter. In the coronary sinus vasculature, dye infusion flows back onto the catheter, providing only a momentary picture of a limited part of the vasculature. The image of dye infusion is captured by retrieving the dye-infusion period from memory and displaying it as a still picture.

Within the first few centimeters into the coronary sinus, about a 90-degree turn is made to enter the coronary sinus branch, which traverses the left ventricle. About 3-6 cm beyond this turn, the posterior vein of the left ventricle branches off in about a 90-degree bend, near the anterior free wall of the left ventricle. It subsequently branches into lateral branches running down to near the anterior apex. Another 4-8 cm beyond the posterior vein branch the coronary sinus becomes the great cardiac vein, which branches in sharp bends to the antero-lateral branches. Both the posterior and antero-lateral branches are candidates for left ventricular pacing or the placement of other devices for treating the left ventricle.

Pacing the left ventricle is accomplished by inserting a lead through the opening (ostium or os) of the coronary sinus and into a distal branch near left ventricular muscle fibers. The difficulties are three-fold:

1. Finding the opening (ostium) of the coronary sinus. Patients in CHF (congestive heart failure) have hypertrophied hearts, which alters the location and size of the coronary sinus. Physicians routinely place leads in the coronary sinus during routine EP (electrophysiologic) studies, however, they are dealing with normal-sized hearts with electrical conduction defects. With CHF patient candidates, finding the opening is much more elusive. Sometimes it is located significantly off-center from the normal location since the heart has hypertrophied. Other times, flaps of tissue prevent entry into the coronary sinus.

2. Advancing the lead through the coronary sinus to a branch in close proximity to the left ventricle so it can be chronically paced. Pacemaker implants are performed on the right side of the heart since implants in the left heart could lead to thrombi heaving deleterious consequences such as a stroke or heart attack. The coronary sinus is the only area of the heart anatomy by which a lead can be inserted from the right heart into close proximity to the left ventricle. In fact, the tip of the pacing lead needs to be within several millimeters of ventricular muscle to successfully pace the ventricle. The coronary sinus branches into segments, five of which traverse the left ventricle. Locating the proper left-ventricular branch (where the left ventricle can be chronically paced) has been difficult in biventricular pacing clinical studies. Hypertrophied hearts also alter the location and length of these branches. Finding the correct branch in these highly variable hearts has been the other major challenge in biventricular pacing.

3. Preventing the lead from dislodging in the first few months following implantation. Since the coronary sinus lead is not anchored in the coronary sinus and it is undergoing significant motion from the left ventricle beating vigorously, these leads have a high dislodgement rate of 10-20%. Dislodgement incidence is reduced if the lead is wedged far enough into a lateral branch of the coronary vein.

There have many disclosures of guide catheters and coronary sinus lead catheters that use pre-formed, deflecting and steerable curves to assist implantation into the coronary sinus os under fluoroscopy. Some disclosures attempt to provide guiding catheters (sheaths) or coronary sinus lead catheters with preferential curves of different flexibilities. This enables the physician to position the catheter near the coronary sinus where it is manipulated to enter the coronary sinus os. Such multi-radius curvature coronary sinus leads include Adams (U.S. Pat. No. 5,433,729), Lurie (U.S. Pat. No. 5,423,772), Tockman (U.S. Pat. No. 6,129,750) and Jaraczewski (U.S. Pat. No. 5,445,148). A variation to these curvatures is Swoyer (U.S. Pat. No. 5,683,445) who teaches a configuration with multiple 45-degree bends to position the electrode closely to the venous wall. Guiding catheters with angled curvatures include Randolph (U.S. Pat. No. 5,775,327), Lurie (USP App US2002/0029030), and Toner (U.S. Pat. No. 5,488,960). A deflectable guide catheter is proposed by Williams (U.S. Pat. No. 6,408,214B1) in which a greater curvature can be achieved by pulling on a handle at the proximal end. A steerable, coronary sinus catheter is proposed by Ockuly (U.S. Pat. No. 6,458,107B1) in which the catheter is curved at steerable angles in one plane.

The purpose of the above inventions is to direct the catheter into the coronary sinus os, not to direct it into the appropriate branch of coronary sinus vasculature. Once in the coronary sinus vasculature, the guiding catheter would need to make approximately two 90 degree bends to reach a site appropriate for left ventricular pacing. Due to variations in the length of the vessels and the degree of bend in the branch points among hypertrophic heart patients, a fixed curved catheter would have the curve points and angles in the wrong place for the majority of patients. Even the deflectable catheter of Williams (U.S. Pat. No. 6,408,214B1) and the steerable, coronary sinus catheter as proposed by Ockuly (U.S. Pat. No. 6,458,107B 1) are intended only to find the coronary sinus os by “touch and feel”, not to navigate in the coronary sinus vasculature through potentially tight, 90 degree bends. The curves described in these patents have a much too high radius of curvature to navigate within the coronary sinus branches usually used for long-term pacing.

Despite the existence of shaped guide catheters and leads, physicians most commonly prefer to use a standard steerable EP ablation catheter, such as described by Avitall (U.S. Pat. No. 5,642,736). These catheters are favored to find the coronary sinus os, even though designed for mapping and ablation purposes, since physicians are familiar with the catheter's characteristics. In this approach, the physician inserts the steerable EP ablation catheter into the right atrium and then applies different curves to the distal end by manipulating controls on the proximal end. The catheter is usually dragged along the atrial wall until it encounters the coronary sinus os. Once the coronary sinus os is entered, a sheath is slid over the EP ablation catheter to cannulate the coronary sinus. The EP ablation catheter is then removed, leaving behind the sheath. The next steps depend on the configuration of the coronary sinus pacing lead. Originally, the lead had no guidewire channel, so once the sheath was in place, the coronary sinus lead was inserted through the sheath and manipulated using an internal stylet to enter the appropriate branch of the coronary sinus. Often, radio opaque dye is infused into the sinus and a snapshot is taken on the fluoroscopy machine to elucidate the branching points within the coronary sinus. Using the coronary sinus lead to access the proper branch was difficult due to the size of the lead and the inability to make sharp-angled bends required to access a suitable coronary sinus branch.

In an effort to have a more navigable catheter, cardiac pacemaker manufacturers developed coronary sinus leads with an open channel through the lead, through which a guidewire could be inserted. This permitted the physician to find the coronary sinus branch with a small flexible guidewire, followed by insertion of the lead over the guidewire. When this system is used, following cannulation by the sheath, the guidewire is then inserted into the sheath and radio opaque dye is infused into the coronary sinus allowing a momentary picture of the coronary sinus vascular tree to be captured by the fluoroscopy camera. The physician then manipulates the guidewire to enter a branch suitable for long-term ventricular pacing. Once a site has been located, the coronary sinus lead is inserted over the guidewire and advanced until it occupies a suitable pacing site. Pacing and sensing thresholds are then taken to verify the coronary sinus lead is positioned to provide long-term left ventricular pacing for the patient. Once in proper position, the guidewire is removed and the lead proximal connector end is connected to the pacemaker.

The complexity in the curve geometries and stiffness characteristics of the above disclosures is due to the physician relying on “touch and feel” at the proximal end of the catheter. The various geometries place the coronary sinus guide catheter or lead in close proximity to the coronary sinus where small manipulations are only required to enter into the coronary sinus os. The difficulty with pre-curved catheters is the extreme variability of coronary sinus location and geometry in hypertrophic hearts. The entire heart and its internal structures tend to be distended by the growth of the heart. In addition, about 20% of the patients have flaps over the coronary sinus, which prevent entry from certain directions. As a consequence of these limitations, implantation of a coronary sinus lead significantly increases the time of pacemaker implantation. A conventional right-sided pacer requires 1-2 hours for implantation with over a 99% success rate. Biventricular pacers require 3-6 hours implantation time, simply because of the difficulty in implanting the coronary sinus lead. Furthermore, the implantation success rate is only 80-90%, with cases abandoned because of inability to implant the coronary sinus lead. Moreover, following the implant, coronary sinus leads are much more prone to lead dislodgement. Reports suggest dislodgement rates of about 10-20% have been observed. Coronary sinus leads dislodge because anchoring means such as tines or screws, commonly used in the right atrium and ventricle, cannot be used in the coronary sinus. Stability is achieved by wedging the lead into a small branch to create a tight fit between the catheter and the coronary sinus branch.

Recently, several real-time forward-imaging technologies have been developed which permit the physician to image the relation of the catheter to the os and branch points in front of the catheter. A forward-viewing technology can be a transducer near the distal end of the catheter, providing a view ahead of the catheter tip. For the purposes of this patent, forward-imaging is defined as imaging at an angle relative to the center axis of the catheter of less than 90 degrees which includes direct as well as off-angle forward imaging. Examples include near-infrared light Amundson (U.S. Pat. No. 6,178,346) and forward-imaging ultrasound such as Lin (U.S. Pat. No. 6,200,269). A forward-imaging technology is also providing local image enhancement at the catheter tip so that whole body real-time imaging can elucidate the relation of the catheter tip to the coronary sinus os or branch. An example is a modification of coronary sinus venography in which a radio opaque dye is infused in the coronary sinus and the heart region viewed with fluoroscopy. If the dye flows out through a lumen in the catheter tip for a long enough duration it becomes forward-viewing since it can be determined from whole body fluoroscopy where the catheter tip is located by observing the flow start point, and the vasculature ahead of the catheter tip. It becomes real-time since articulations of the catheter tip can be observed in the fluoroscopy monitor. Since the coronary sinus expels blood, the dye remains in the coronary sinus vasculature for only a brief instant and captured by the fluoroscopy camera. Recent developments include using a balloon expanded inside the os entrance to prolong the time for the dye to diffuse back into the right atrium. Another example is magnetic resonance imaging with an internal magnetic coil in or around the catheter. The internal coil highlights the catheter region when viewed with a whole body magnetic resonance imager, providing images of the catheter position and branch points in the coronary sinus. Magnetic resonance imaging systems are currently too slow to view in real-time, although future improvements may eventually render it a real-time imaging modality.

Forward-imaging technologies in the form of a transducer in the catheter tip include disclosures by Amundson (U.S. Pat. No. 6,178,346) using near-infrared light, forward-viewing ultrasound such as Lin (U.S. Pat. No. 6,200, 269), optical coherence tomography such as Wang (U.S. Pat. No. 6,041,248) and optical coherence domain reflectometry as described by Zeylikovich (U.S. Pat. No. 6,437,867). When a forward-imaging technology is included in the coronary sinus/branch-seeking catheter, different design considerations apply since the physician manipulates the catheter while observing an image on a monitor, which displays the structures in front of the catheter. This is most clearly demonstrated with near-infrared imaging (U.S. Pat. No. 6,178,346) in which a direct image is obtained, through blood, of the structures in the lower right atrium. This system uses near-infrared light above 800 nm to permit viewing through blood. Wavelengths between 1500-1900 nm are particularly advantageous since scattering and absorption are low in this wavelength region. Light is reflected off of the structure viewed, returning to the catheter where the reflected light is collected and transmitted to an infrared camera.

When near-infrared imaging is employed, the inferior vena cava appears as a large hole, and the coronary sinus appears as a smaller hole. The tricuspid valve appears as a large hole with valve leaflets. Using these and other markers, a physician can direct the catheter so it is centered over the coronary sinus, and then push it through the coronary sinus os. Once in the coronary sinus, branches would appear as bifurcations and two holes would be visible. Using other forward-viewing technologies, with image enhancement of holes present, could provide a similar picture.

Another real-time, forward-imaging technology that is somewhat different in nature is a lead navigation system such as the CARTO system manufactured by Biosense Webster. Such a system shows the relationship of the catheter tip to the cardiac structure of interest. The Biosense/Webster system provides the six coordinates (x, y, z, yaw, pitch and roll) of a catheter containing a magnetic element. By dragging this catheter on the cardiac interior, while simultaneously recording the electrical potentials at each point, a map of the cardiac interior can be obtained. Objects such as holes are recognized from the absence of electrical potentials and can be displayed as pictorial representations. The image, in this case, shows the catheter position relative to the coronary sinus.

In addition to this system, Medtronics manufacturers a lead locater system based on impedance, and Boston Scientific has one based on ultrasound. All systems require a locatable element in the catheter. In contrast to coronary sinus venography which can only image in the coronary vascular tree, systems like CARTO would only be useful in finding the coronary sinus os. These systems would not be useful in the coronary sinus vasculature, since the mapping catheter must first be in the vicinity of a structure to allow the system to render an image of the structure. These systems are useful only as feedback for finding the coronary sinus. However, in that respect they are no different than other feedback systems; they provide a real-time image of the relation of the catheter tip to the coronary sinus os. Manipulations can be observed in the image. These systems have not been employed to place coronary sinus catheters because of the length of time it takes to map the right atrium. More automated mapping may make these technologies candidates for feedback in coronary sinus catheter placement.

Visual feedback radically alters the design considerations for guide catheters. The disclosures Adams (U.S. Pat. No. 5,433,729), Lurie (U.S. Pat. No. 5,423,772), Tockman (U.S. Pat. No. 6,129,750) and Jaraczewski (U.S. Pat. No. 5,445,148) all teach catheters which are designed with advantageous angled segments and flexibilities so that manipulation under fluoroscopy will successfully cannulate the coronary sinus os. When the coronary os and branches are imaged, cannulation can be easily accomplished with a deflectable catheter—if the physician has direct feedback about whether his manipulations are bringing him closer to or further from the coronary sinus os/branches as he is viewing the image during the manipulations.

### SUMMARY OF THE INVENTION

The object of the invention is to provide a method and a coronary sinus access catheter system that simplifies the insertion of leads and other catheters into the os and distal branches of the coronary sinus using forward-imaging to assist catheter tip positioning. Forward imaging allows the catheter/sheath to be seen in relation to the hole it is entering, be it the coronary sinus os or branch point within the coronary sinus vasculature. As the catheter is articulated, the forward image provides feedback about its proximity to the structure to be entered. Articulation is accomplished either with a deflection mechanism or by rotating a fixed-curve catheter . As the real-time image is observed, the catheter is centered near the os of the coronary sinus by engaging the deflection mechanism on the proximal end of the catheter or positioning the end of a fixed curve guide catheter. As the catheter is pushed through the os, the forward-viewing imager provides immediate verification of entry into the os. As it continues in the coronary sinus vasculature, branch points are imaged and the catheter usually needs to make several tight-radius bends to enter an appropriate distal branch. Radius of curvatures of about 6-15 mm are required for navigation into lateral branches at near right angles to the main branch. To accomplish this in a deflectable catheter, the distal end of the catheter needs to bend about about 60 degrees over the last centimeter. Similarly, a fixed-curve catheter over the last centimeter of the distal end must have a fixed angle to navigate tight branches.

The tight-radius deflection mechanism consists of one or two deflection wires pulling on a segment of the distal portion of the lead, creating deflections of about 60 degrees over the last centimeter of the catheter distal end. If two wires are used the deflection is bi-directional; one wire creates unidirectional deflections. If unidirectional deflection is used, the catheter can be torqued so that rotation on the proximal end results in a similar rotation on the distal end. The combination of rotating and deflecting permits the physician to navigate in 360 degrees about the catheter axis. The bi-directional system has the advantage of requiring less rotation to orient the catheter; the unidirectional deflection mechanism allows in a smaller catheter since only one wire is needed in the catheter. The deflection wire(s) is connected to a handle on the proximal end, which when manipulated, deflects the tip of the catheter.

A fixed-curved catheter must be torqueable and needs to have sufficient flexibility and angle on the distal end to navigate tight-radius turns. As the branch point is viewed in the imager, the catheter is rotated and pushed to enable the catheter to catch the lip of the desired branch. This is most easily achieved with a catheter, which is flexible on the last few centimeters of the distal end and at a fixed angle such as 30 degrees or greater. Such a catheter can be pushed and rotated to create greater angles in the coronary sinus vasculature and navigated to tight branch points.

In the preferred embodiment used for placing coronary sinus leads, the system consists of a multi-lumen catheter containing lumens for the forward-viewing near infrared transducer, a guidewire channel and a deflection wire connected to a forward-viewing near infrared imaging (U.S. Pat. No. 6,178,346) acquisition unit. The acquisition unit contains the near infrared light source and the infrared camera, a system controller, and an interconnect cable for connection to the disposable catheter. The multi-lumen catheter has one lumen, about one mm in diameter for the illumination and collection fibers of the near infrared forward-viewing transducer, another lumen about 0.5 mm in diameter and a lumen for a steering wire which is also about 0.5 mm in diameter. Ideally, the overall catheter diameter is 2.3 mm (7 French) or smaller. The catheter is inserted with a sheath into the right atrium, where the catheter or sheath is articulated or manipulated to bring the coronary sinus os into view. The catheter is directed through the os using feedback from the near-infrared transducer and deflecting the catheter from a controller at the proximal end of the catheter or manipulating a fixed-curve guide catheter. As the catheter navigates through the coronary sinus vasculature, images of the branch points appear in the forward-viewing monitor and the catheter is deflected to advance into the proper branch. Once the catheter is inserted to the appropriate branch point, a guidewire is inserted to the distal end of the catheter, and the catheter is removed and a coronary sinus lead inserted over the wire to the distal branch. If an acceptable position has been reached by pacing threshold verification and stability considerations, the guidewire is then removed and the coronary sinus lead implanted in the biventricular pacemaker.

In another coronary sinus lead placement embodiment, balloon-augmented coronary sinus venography is used for forward viewing. The tight-radius deflecting catheter consists of a two-lumen device, a small lumen for the deflection wire, and another lumen for infusion of fluoroscopic dye and for passage of a guidewire. This system has limited usefulness in finding the os of the coronary, sinus, but is useful in the coronary sinus vasculature if modified to produce longer duration pictures. The pictures need to be of long enough duration and frequent enough to permit the physician to view his manipulations on the fluoroscopic monitor. This is accomplished by having a balloon on a sheath, through which the catheter is inserted. In addition, the dye infusion in controlled from an infusion pump activated by a foot switch. The high-pressure dye lumen has a flow restrictor on the distal end of the catheter to propel the dye farther up the coronary sinus vasculature. The sheath catheter assembly is inserted into the coronary sinus with the balloon inside the os. Inflation of the balloon with a saline solution minimizes back leakage of the dye infusion. In one embodiment, as the catheter is advanced in the coronary sinus vasculature, puffs of dye are infused through the dye lumen by foot switch activation by the physician as he is threading the catheter in the coronary sinus vasculature The balloon may also be expanded prior to the dye puff and kept expanded for the expected time for the dye to be diffused into the right atrium. The result is a series of short-duration images showing the catheter distal end where the dye starts flowing and its position relative to the coronary sinus branching point he is navigating. As each branch point is encountered the physician deflects the catheter to permit entry into the proper branch. Once the catheter is inserted to the appropriate branch point, a guidewire is inserted to the distal end of the catheter through the dye lumen, the catheter is removed, leaving the wire in the distal branch. If an acceptable position has been reached by pacing threshold verification and stability considerations, the guidewire is then removed and the coronary sinus lead implanted in the biventricular pacemaker.

### BRIEF DESCRIPTION OF THE DRAWINGS

FIG. 1 is a drawing of a catheter according to the present invention in the coronary sinus vasculature

FIG. 2A is a drawing of a catheter encountering a lateral venous branch point at a 90-degree angle with respect to the main branch.

FIG. 2B is a drawing of a catheter encountering a lateral venous branch point at a 80-degree angle with respect to the main branch.

FIG. 2C is a drawing of a catheter encountering a lateral venous branch point at a 60-degree angle with respect to the main branch.

FIG. 3 is a system drawing of the preferred embodiment of near-infrared imaging showing the catheter and the near-infrared acquisition system.

FIG. 4 is a drawing illustrating a bend in the catheter according to the present invention.

FIG. 4A is a drawing of the handle portion of the catheter used in a preferred embodiment using near-infrared light.

FIG. 4B is a drawing of the cross-section of the catheter.

FIG. 5 is a system drawing of an embodiment using coronary venography, showing the catheter and the coronary venography infusion system.

FIG. 6 is a drawing of the cross-section of the coronary venography catheter embodiment.

FIG. 7 is a drawing of the catheter in a near-infrared imaging embodiment showing a cross-section of the catheter embodiment where the coronary sinus lead is inserted through a port of the catheter.

### DETAILED DESCRIPTIONS OF THE EMBODIMENTS

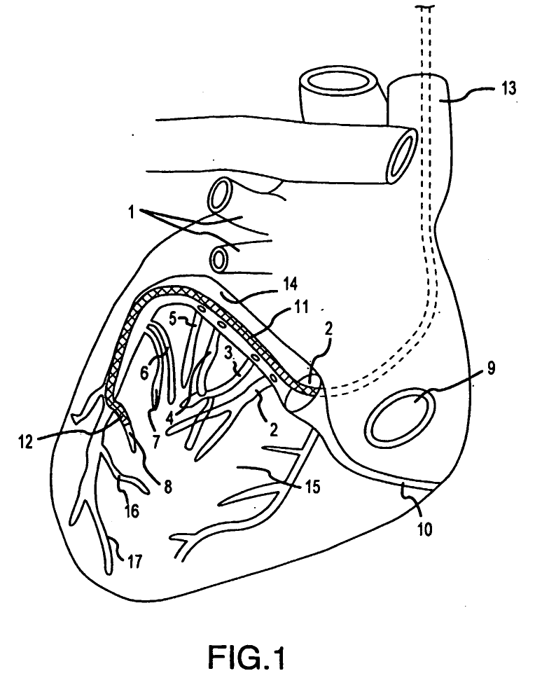

FIG. 1 shows the expected route of the catheter (11) as it is inserted onto the coronary sinus to a position in the anterior-lateral branch (8) of the coronary sinus vasculature. A sinus lead (12) is inserted through a puncture or cutdown technique into the subclavian vein where it eventually enters the superior vena cava (13). The lead is directed to the tricuspid valve plane in the lower right atrium where the os of the coronary sinus (1) is located. The os of the coronary sinus (1) is located near the tricuspid valve and the inferior vena cava (9). After entry into the coronary sinus os, the coronary sinus diverges into the great cardiac vein (14) and the right coronary vein (10). Directing the catheter (11) in the direction of the great cardiac vein (14) requires a tight radius deflection towards the left side of heart. As the catheter traverses the coronary sinus (1), several branch points called the posterior lateral coronary veins (2,3,4,5) run laterally along the left ventricle (15). As the catheter traverses past the posterior lateral coronary veins (2,3,4,5), the coronary sinus becomes the great cardiac vein (14) where more lateral branches are found (6,7). Descending farther down the great cardiac vein (14) the anterior-lateral branches (8, 16, 17) are found. In this figure, the lead is positioned in the first anterior-lateral branch (8). In fact, any of the lateral branches (2-8, 16, 17) are preferred sites for implantation of the coronary sinus lead (12). Entry into many of these branches requires tight radius curvature.

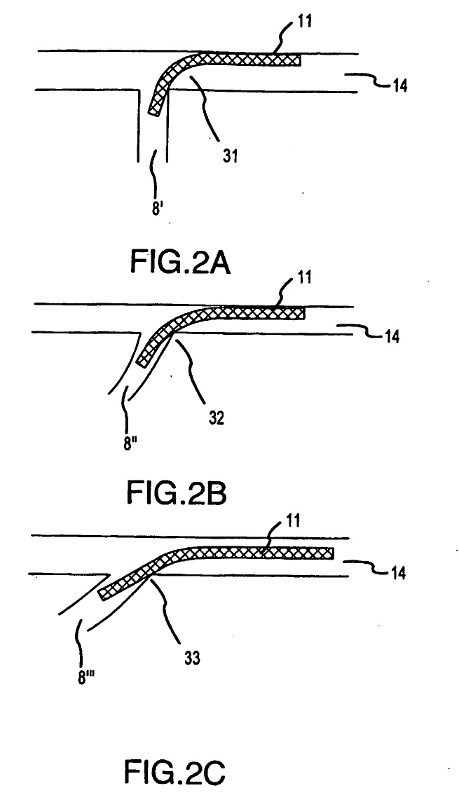

FIG. 2 shows a catheter (11) encountering three lateral branches (8′; 8″, 8′″) from the great cardiac vein (14) of differing angles (2A, 2B, 2C) with respect to the great cardiac vein (14). FIG. 2A shows a catheter (11) encountering a branch (8′) at a 90-degree angle from the great cardiac vein (14). A deflection with a radius of curvature (31) of about 6 mm or less is required to reach this branch. This amounts to about a 60-degree bend over the last centimeter of the distal end of the catheter. FIG. 2B shows a catheter (11) encountering a branch (8″) at about an 80-degree angle from the great cardiac vein (14). A deflection with a radius of curvature (32) of about 9 mm or less is required to reach this branch. This amounts to about a 45- degree bend over the last 1.5 centimeters of the distal end of the catheter. FIG. 2C shows a catheter (11) encountering a branch (8′″) at a 45-degree angle from the great cardiac vein (14). A deflection with a radius of curvature (33) of about 15 mm or less is required to reach this branch. This amounts to about a 30-degree bend over the last two centimeters of the distal end of the catheter. In these examples, only the branch shown in FIG. 2C might be navigable with conventional deflection catheters such as those used in EP/ablation catheters. FIGS. 2A and 2B are examples of sharp bends in the coronary sinus lateral veins, requiring a deflecting mechanism with a short radius of curvature of under one centimeter.

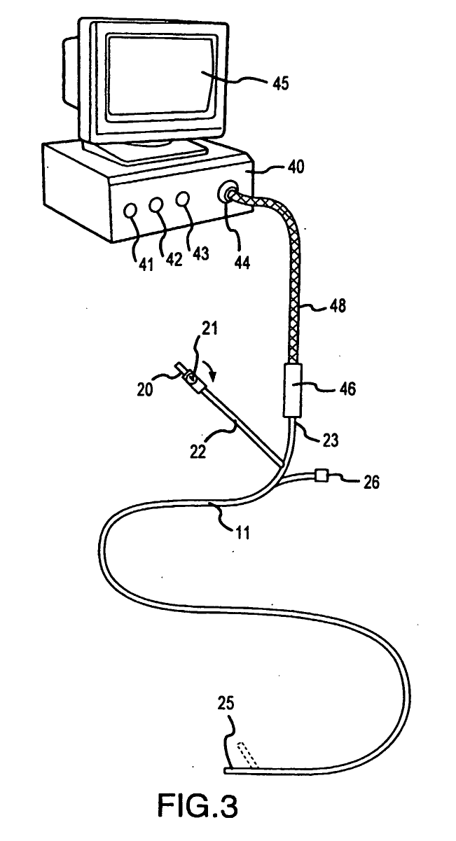

The system for the preferred embodiment, using near-infrared imaging as feedback, is shown in FIG. 3. Near-infrared imaging is ideal since it produces direct visualization of the structures ahead of the catheter. The system consists of a multi-lumen catheter (11) with a bifurcated proximal end, one end (22) containing the steering wires and connected to a handle (20) containing a knob (21) which when turned deflects the tip of the catheter (25). The other bifurcation at the proximal end (23) of the catheter (11) contains the optical fibers used in the near-infrared imaging. It is connected to an interface box (46) containing the light source (such as a diode) and imaging sensor (such as including an IR camera). A cable (48) connects box (46) to the near-infrared imaging acquisition unit (40) as described in U.S. Pat. No. 6,178,346. The acquisition unit (40) contains the system controller and image processing software and imaging controls (41, 42, 43). The details of the infrared-imaging are described in U.S. Pat. No. 6,178,346 and thus need not be repeated in detail herein. Briefly summarizing that patent, the catheter 11 tip 25 houses an optical head assembly which, in connection with light source, imaging sensor, and associated components enable infrared catheter imaging.

In an alternative embodiment, the catheter tip 25 houses a transducer which allows for imaging either through electromagnetic energy, including magnetic energy, or through intraluminal or intracavity ultrasound. The infrared, electromagnetic, or ultrasound energy imaging techniques are incorporated into every embodiment disclosed herein.

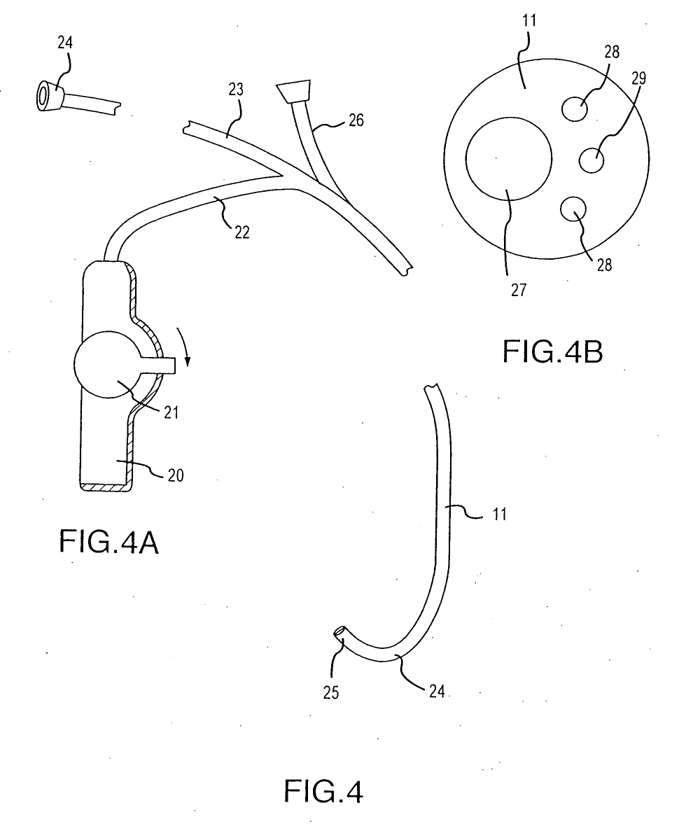

The multi-lumen catheter (11) has one larger lumen (27), about one mm in diameter for the illumination and collection fibers of the near infrared forward-viewing transducer, another small lumen (29) about 0.5 mm in diameter and two lumens (28) for steering wires about 0.3 mm in diameter. The overall catheter diameter is 2.3 mm (7 French) or smaller. FIG. 4 shows that the catheter (11) steering enables tight-radius deflections occurring at a point (24) about one cm from the distal end of the catheter (25). The catheter tip (25) can be deflected about 60 degrees or more, by turning the knob (21) on the handle (20). The catheter is inserted with a deflectable or fixed-curved sheath into the right atrium, where the sheath is deflected and pushed or otherwise manipulated to bring the tricuspid plane of the right atrium into view. The catheter tip (25) is deflected to bring the coronary os into view. The catheter (11) is then pushed through the coronary sinus os. All of the deflections are made using feedback from the imaging information and deflecting the catheter from the knob (21) in the handle (20) of the catheter. As the catheter navigates through the coronary sinus vasculature, images of the branch points appear in the forward-viewing monitor and the catheter tip (25) is deflected to advance into the proper branch. Once the catheter is inserted to the appropriate branch point, a guidewire is inserted in the guidewire channel (29) at the proximal end (26) of the catheter (11). The catheter is removed and a coronary sinus lead inserted over the wire to the distal branch. If an acceptable position has been reached by pacing threshold verification and stability considerations, the guidewire is then removed and the coronary sinus lead implanted in the biventricular pacemaker.

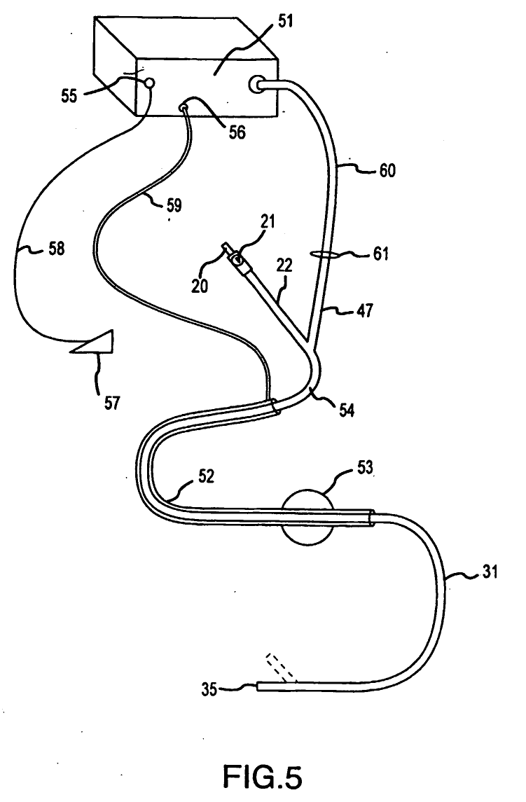

FIG. 5 shows a coronary sinus lead placement embodiment with an automated balloon-augmented coronary sinus venography feedback control. As the catheter is inserted into the right atrium and into the coronary sinus and forward imaging is desired, activation of the foot switch (57) expands the balloon (53) with a saline solution from an infusion pump (51) to reduce the coronary sinus outflow rate and infuses radio opaque dye. The infusion pump (51) also infuses radio opaque dye into a high pressure tube (47) containing a flow restrictor at the distal end (35), such as a series of holes, to increase the pressure and propel the radio opaque fluid farther into the coronary venous system. The balloon (53) remains inflated for a short period until the dye is diffused out of the coronary sinus at which point it deflates, permitting blood flow to return to the coronary sinus. Alternatively, balloon inflation and dye infusion could be automatically activated on a frequent basis to provide intermittent real time imaging.

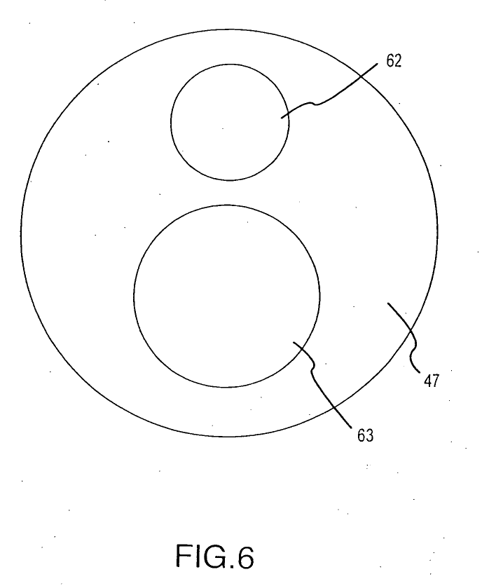

The system consists of a deflectable catheter (31) enclosed in a sheath (52) containing an expandable balloon (53). The catheter bifurcates to a steering portion (22) connected to a handle (20) with deflection accomplished by turning the handle knob (21). The other bifurcation is a high-pressure tube (47) connected to a connector (61), which is, in turn, connected to the infusion pump (51). FIG. 6 shows the cross-section of the high-pressure tube (47) and its lumens. The tight-radius deflecting catheter consists of a dual -lumen device, a small lumen (62) for a unideflection mode deflection wire, and a larger lumen (63) for infusion of fluoroscopic dye and for passage of a guidewire. The system could also have separate lumens for the guidewire and the dye infusion. This system would have limited usefulness in finding the coronary sinus, but would be useful, in the coronary sinus vasculature if the system could be modified to produce longer duration pictures. The pictures need to be of long enough duration and frequent enough to permit the physician to view his manipulations on the fluoroscopic monitor. This is accomplished, by a footswitch (57), which both activates the occlusive balloon and initiates dye infusion. Alternatively, the activation of the occlusive balloon and dye infusion could be performed automatically at a fixed time interval. Using either method, the result is a series of short-duration images showing the catheter distal end where the dye starts flowing and its position relative to the coronary sinus branching point he is navigating. As each branch point is encountered the physician deflects the catheter to permit entry into the proper branch. Once the catheter is inserted to the appropriate branch point, a guidewire is inserted to the distal end of the catheter through the dye lumen (63), the catheter is removed, leaving the wire in the distal branch. If an acceptable position has been reached by pacing threshold verification and stability considerations, the guidewire is then removed and the coronary sinus lead implanted in the biventricular pacemaker.

Another embodiment uses the same near-infrared imaging system as the first embodiment except the entire coronary sinus lead is inserted through a port instead of the guidewire. Referring to FIG. 3, the system consists of a multi-lumen catheter (11) with a bifurcated proximal end, one end (22) containing the steering wires and connected to a handle (20) containing a knob (21) which when turned deflects the tip of the catheter (25). The other bifurcation at the proximal end of the catheter (23) contains the optical fibers used in the near-infrared imaging. It is connected to an interface box (46) containing the light source and imaging sensor. The interface box (46) is in turn connected by a cable to the near infrared imaging acquisition unit (40) as described in U.S. Pat. No. 6,178,346. The acquisition unit (40) contains the system controller and image processing software and imaging controls (41, 42, 43). The catheter (11) steering enables tight-radius deflections occurring at a point (24) (see FIG. 4) about one cm from the distal end of the catheter (25). The catheter tip (25) can be deflected about 60 degrees by turning the knob (21) on the handle (20).

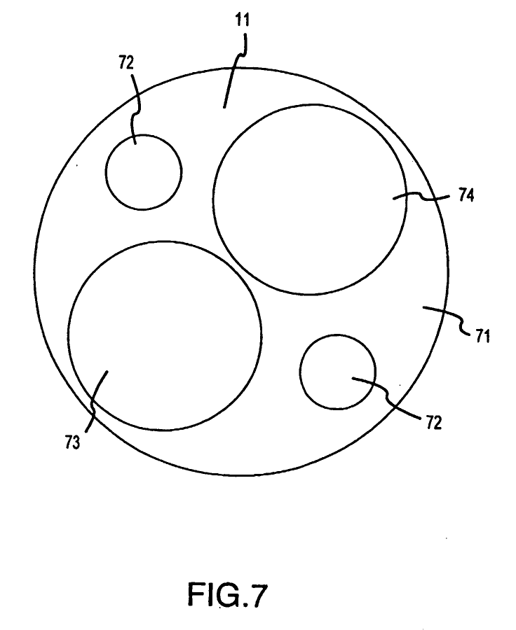

The catheter is inserted with a deflectable or fixed-curve sheath into the right atrium, where the sheath is deflected and pushed to bring the tricuspid plane of the right atrium into view. The catheter tip (25) is deflected or manipulated to bring the coronary os into view. The catheter (11) is then pushed through the coronary sinus os. All of the deflections are made using feedback from the near-infrared transducer and deflecting the catheter from the knob (21) on the handle (20) of the catheter. Referring to FIG. 7, the multi-lumen catheter (11) has a larger lumen (73), about one mm in diameter for the illumination and collection fibers of the near infrared forward-viewing transducer, another large lumen (74) about 1.3 mm in diameter and two lumens (72) for steering wires about 0.3 mm in diameter.

As the catheter navigates through the coronary sinus vasculature, images of the branch points appear in the forward-viewing monitor and the catheter tip (25) is deflected to advance into the proper branch. Once the catheter is inserted to the appropriate branch point, the coronary sinus lead is inserted in the guidewire channel (74) at the distal end of the catheter. The catheter is removed and a coronary sinus lead remains in the distal branch. The lead can be tested for proper pacing threshhold and stability considerations with the catheter still in place. If an acceptable position has been reached by pacing threshold verification and stability considerations, the coronary sinus lead connected to the biventricular pacemaker.

From the foregoing, it should be appreciated that a method has been invented for finding the coronary sinus os and/or navigating the coronary sinus branches based on a catheter employing forward, real-time imaging. The coronary sinus os can be entered by using a deflectable or fixed-curve catheter with manipulations under view by the real-time, forward-imaging system. The coronary sinus branches can be selected by using a deflectable (torqueable) or a preferentially-curved, floppy-tip catheter, guided by the real-time forward imaging system Moreover, the embodiments demonstrate the invention of a deflectable catheter, using real-time, forward imaging for guidance, which can be navigated to distal coronary sinus branches for the delivery of devices such as guidewires and cardiac pacing leads.

What is claimed is:

1. A method of cannulating the coronary sinus os comprising the steps of:
inserting a catheter into the right atrium,
providing a means in the catheter to permit the generation of real-time forward imaging of the relation between catheter tip and the surrounding tissue,
deflecting or manipulating the catheter using guidance from the images to move the catheter into the coronary sinus os

2. The method of claim 1, where the catheter has a fixed-curve and is torqueable

3. The method of claim 1, where the catheter has a floppy tip

4. The method of claim 1 where the means of creating real-time forward imaging includes an optic head assembly.

5. The method of claim 4, wherein the optic assembly uses infrared light for imaging.

6. The method of claim 1 where the means of creating real-time forward imaging includes a transducer.

7. The method of claim 1, where the means of providing real-time forward imaging is by determining catheter location from an element in the catheter and creating a map of the right atrium,

8. The method of claim 7, where the element is electromagnetic.

9. The method of claim 8, where the element emits ultrasound energy.

10. A method of navigating the coronary sinus branches in coronary sinus vaculature having an os comprising the steps of:
inserting a catheter into the coronary sinus os;
advancing the catheter to a branch point in the coronary sinus vasculature;
providing a means in the catheter to permit the generation of real-time forward images of the relation between catheter tip and the branch point; and
deflecting or manipulating the catheter using guidance from the images to move the catheter into the coronary sinus branch.

11. The method of claim 10, wherein the catheter is a fixed-curve and torqueable.

12. The method of claim 10, wherein the catheter is a floppy tip

13. The method of claim 10 wherein the means of generation of real-time forward images uses infrared illumination.

14. The method of claim 10 wherein the means of generation of real-time forward images uses a transducer.

15. The method of claim 10 wherein the transducer emits ultrasound energy.

16. The method of claim 10 wherein the transducer emits infrared energy.

17. The method of claim 10, wherein the means of providing real-time forward images is whole-body fluoroscopy of controlled dye infusion through a distal end of the catheter.

18. The method of claim 17, wherein the dye infusion is controlled by an infusion pump.

19. The method of claim 17, wherein an occlusive balloon limits the dye infusion backflow into the right atrium.

20. The method of claim 19, wherein the balloon is expanded only during dye infusion and released following backflow of the dye into the right atrium.

21. The method of claim 20, wherein an infusion pump expands the balloon.

22. The method of claim 17, wherein footswitch activation controls the dye infusion.

23. An access catheter system for insertion into the coronary sinus vasculature comprising:
an elongated catheter body having a tip capable of navigating the sharp turns of the coronary sinus vasculature and incorporating means in the catheter for generation of real-time forward imaging of the relation between the catheter tip and the surrounding tissue
an open lumen for the passage of at least one medical device;
an acquisition unit to process the images; and
a monitor to display real-time images.

24. The catheter of claim 23, wherein the catheter is deflectable.

25. The catheter of claim 23, wherein the catheter has a fixed-curve and is torqueable.

26. The catheter of claim 23, wherein the catheter has a floppy tip.

27. The catheter of claim 23, wherein the catheter tip has a radius of curvature under 15 mm.

28. The catheter of claim 27, wherein the catheter tip deflection has a radius of curvature under 10 mm.

29. The catheter of claim 23, wherein the means for generation real time forward imaging includes near infrared imaging.

30. The catheter of claim 29 wherein the means for generation includes an optical head assembly in the catheter tip.

31. The catheter of claim 23, where the beams for generation include an ultrasound or elecromagnetic transducer in the catheter tip.

32. An access catheter system for insertion into the coronary sinus vasculature comprising:
an elongated catheter body with a tip capable of navigating the sharp turns of the coronary sinus vasculature and incorporating means in the catheter to permit the generation of real-time forward imaging of the relation between the catheter tip and the surrounding tissue;
a channel with a flow restrictor for the controlled infusion of radio opaque dye;
a radio opaque dye infusion apparatus;
a open lumen for the passage of at least one medical device; and
a fluoroscopy machine for viewing dye infusion.

33. The catheter of claim 32, wherein the catheter is deflectable.

34. The catheter of claim 32, wherein the catheter has a fixed-curve and is torqueable.

35. The catheter of claim 32, wherein the catheter has a floppy tip.

36. The catheter of claim 32 wherein the flow restrictor is a series of holes.

37. The catheter of claim 32 wherein the catheter tip is capable of a radius of curvature under 15 mm.

38. The catheter of claim 37 wherein the catheter tip is capable of a radius of curvature under 10 mm.

39. The catheter of claim 32 wherein the radio opaque dye infusion apparatus includes an infusion pump.

40. The catheter of claim 32, wherein an occlusive balloon limits the dye infusion backflow into the right atrium.

41. The catheter of claim 40, wherein the balloon is expanded only during dye infusion and released following backflow of the dye into the right atrium.

42. The catheter of claim 41, wherein an infusion pump expands the balloon.

43. The catheter of claim 39, wherein the infusion pump is activated by a foot switch.

44. The catheter of claim 43 wherein the infusion pump is activated at a fixed interval.

45. A coronary sinus venography system comprising:
a catheter inserted into a coronary sinus os and containing an occlusive balloon;
an infusion pump for controlling the infusion of radio-opaque dye and saline injection into the catheter and subsequently into the coronary sinus vasculature;
timing the balloon inflation so that the balloon is occluded prior to dye injection; and
a fluoroscopy machine for viewing dye infusion.

46. The catheter of claim 45, wherein the infusion pump is activated by a foot switch.

47. The catheter of claim 45, wherekn the infusion pump is activated automatically at a fixed interval.
